(19) United States
(12) Patent Application Publication
(10) Pub. No.: US 2012/0016189 A1
(43) Pub. Date: Jan. 19, 2012

(54) MALE ANATOMICAL ENHANCEMENT GARMENT

(76) Inventor: Bruce Victor Gioia, Vancouver (CA)

(21) Appl. No.: 13/180,472

(22) Filed: Jul. 11, 2011

Related U.S. Application Data
(60) Provisional application No. 61/365,342, filed on Jul. 18, 2010.

(51) Int. Cl. A61F 5/41

(52) U.S. Cl. 600/41

(57) ABSTRACT

The present invention is a male anatomical enhancement garment to enhance male genitalia with a base, a penis, a scrotum and testicles and a penis head that includes a base band to receive the genitalia that wraps around the base of the genitalia to secure the garment in place, a penis sheath to receive the penis that expands and contracts as the penis expands and contracts and a frontal aperture disposed on the end of the penis sheath that the inserted penis is inserted through. The garment also includes an internally embedded silicon polymer bead or attached elasticized band that is positioned behind the penis head after the penis is received through the front aperture and a pouch that contains the scrotum and testicles. There is also a glans cover that is provided to cover the penis head as well in another embodiment.

This application claims priority to U.S. Provisional Application 61/365,342 filed on Jul. 18, 2010, the entire disclosure of which is incorporated by reference.

### TECHNICAL FIELD & BACKGROUND

Small penis syndrome is a rapidly escalating problem that is having a very destructive psychological impact on contemporary man's sense of masculinity and male identity. This syndrome is primarily related to a relatively normal sized penis that can become smaller than average in the flaccid state. Individuals who experience lack and want for greater genital mass often become deeply embedded in a wide range of psycho-emotional inferiority complexes. These manifest in the most extreme cases with debilitating consequences. There is ample evidence in the medical literature and in online accounts where personal anecdotes of men describe a loss of interest not only in relationships with women but also with life itself as a result of their intense dissatisfaction with penis size.

This complex is becoming even more of a problem due to the “size matters” stigma that is being aggressively promoted in the media particularly in combination with the easy access now available to online pornography sites which for the most part are exclusively promoting male actors with extra-ordinarily large penises. This has had an immensely negative impact on the male perception of masculinity and thus his own sense of identity and self worth. The feelings of inadequacy associated with this complex are to a large degree resolved with the implementation and regular use of this new garment that provides not only support but also augmentation and passive genital exercising properties. This leads to a dramatic increase in self confidence on the psychological level and a more relaxed style of body language on the physical level as well.

It is an object of the present invention to provide an anatomical enhancement garment that utilizes folded or rolled back fabric to create a thick band of material that secures the garment in place which also acts as a collar for grasping the garment when putting-on or removing the garment.

It is an object of the present invention to provide an anatomical enhancement garment that utilizes a thick band of material that secures at the base of the genitals manufactured to specific size configurations which are permanently fixed in place and worn without rolling or folding back the band to fit the garment to wear.

It is an object of the present invention to provide an anatomical enhancement garment that utilizes “displace and replace” principals applied by the thick band of folded, or rolled down material that is fixed in place so that it secures at the base of the genitals thereby effectively pulling and squeezing the flesh away from the body (displace) which subsequently is filled up again with the bulk of fabric (replace) for enhanced size and volume of the male genitalia

It is an object of the present invention to provide an anatomical enhancement garment that utilizes these same displace and replace principals along a penis shaft to apply maximum extension and at the same time to provide the appearance of added girth to a flaccid penis with the bulk fabric of the garment.

It is an object of the present invention to provide an anatomical enhancement garment that utilizes a completely open frontal aperture with a bead of soft silicon or similar type polymer compound embedded directly into the fabric itself to secure the penis glans from retracting back inside the penis sheath portion of the garment. This bead of silicon fits in such a way that it also keeps the glans safely semi-engorged with blood for additional size gains. The open aperture allows for easy donning of the garment and hassle free convenience during urination.

It is an object of the present invention to provide an anatomical enhancement garment that utilizes a completely open frontal aperture with an attached elasticized band which is secured behind the penis glans thereby preventing retraction of the penis into the sheath portion of the garment. The attached band fits in such a way that it also keeps the glans safely semi-engorged with blood for additional size gains. The open aperture allows for easy donning of the garment and hassle free convenience during urination.

It is an object of the present invention to provide an anatomical enhancement garment that utilizes a telescoping or accordion like action that can be implemented to varying degrees in any one design, more or less depending on the level of extension forces that are to be applied at the sheath. The telescoping action takes place along the penis shaft beginning at the base of the sheath portion and ending at the thick band which secures at the base of the genitals where they attach to the body. This telescoping action allows for the natural penis size fluctuations to a limited extent that maintains both comfort and functionality.

### BRIEF DESCRIPTION OF THE DRAWINGS

The present invention will be described by way of exemplary embodiments, but not limitations, illustrated in the accompanying drawing in which like references denote similar elements, and in which:

FIG. 1A illustrates a front side environmental perspective view of an anatomical enhancement garment, in accordance with one embodiment of the present invention.

FIG. 1B illustrates a side perspective view of an anatomical enhancement garment, in accordance with one embodiment of the present invention.

FIG. 1C illustrates a side perspective view of a penis sheath and thick band without pouch and telescoping section of an anatomical enhancement garment, in accordance with one embodiment of the present invention.

FIG. 2 illustrates a side perspective view of an anatomical enhancement garment, in accordance with one embodiment of the present invention.

FIG. 3 illustrates a side perspective view of an anatomical enhancement garment, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

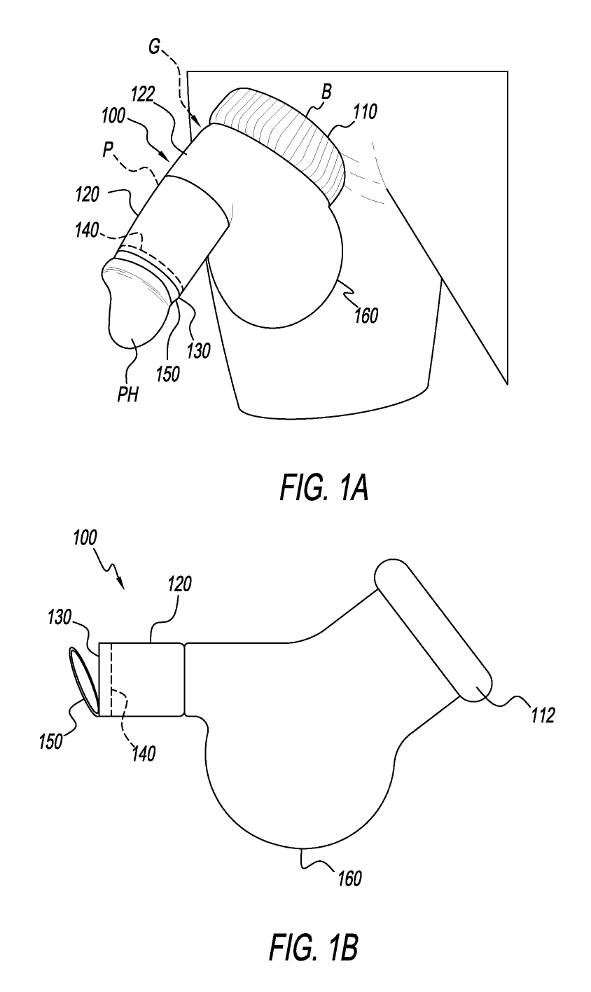

FIG. 1A illustrates a front side environmental perspective view of an anatomical enhancement garment 100, in accordance with one embodiment of the present invention. The anatomical enhancement garment 100 includes a base band 110, a penis sheath 120, a frontal aperture 130, an internally embedded silicon bead 140, an elasticized band 150, and a pouch 160. The anatomical enhancement garment 100 can be made of cloth or any other suitable type of material or in some cases entirely of latex rubber or other soft polymer type compound.

The base band 110 receives the male genitalia G and wraps around the base B of the genitalia G. The base band 110 is created out of cloth fabric material or soft polymer type compound by forming a thick ring or collar when it is folded, rolled down or otherwise permanently manufactured in place which serves to secure the anatomical enhancement garment 100 in place at the base of the genitals while simultaneously applying the forces of the displace and replace principals. The base band 110 also conveniently acts as a handle to grip with the fingers inside of the anatomical enhancement garment 100 to aid in donning the garment. The penis sheath 120 receives a user's penis P and expands as the penis P expands and grows as needed to accommodate both the flaccid and erect penis. The penis sheath 120 is also made of relatively thicker material to provide various degrees of extension capabilities depending on the specification of any one design. The thinner section 122 allows the material to remain flexible so as to retract and fold within itself in a telescopic or accordion like manner. The frontal aperture 130 receives a user's penis via insertion through the penis sheath 120 from inside the garment while the genital mass is secured at its base B adjacent to the user's body with the base band 110.

The internally embedded silicon polymer bead 140 or elasticized band 150 is positioned behind the penis head PH after the penis is received through the front aperture 130 to positively secure the anatomical enhancement garment 100 in place. The elasticized band 150 may or may not include a drawstring type feature (not shown) which can be concealed within the garment itself.

The pouch 160 contains the scrotum and testicles of the user (not shown) and is located adjacent to the base band 110. The base band 110 is placed over the entire genital structure by first putting the scrotum and testicles inside the pouch 160. At the same time the penis is inserted into the base of the penis sheath 120 from the inside. The genitals are then shaken to automatically extend the penis head through the frontal aperture 130. The silicon bead 140 or elasticized band 150 is then positioned behind the penis head to prevent unintentional retraction of the same back inside the sheath portion 120 of the anatomical enhancement garment 100 when in use.

FIG. 1B illustrates a side perspective view of an anatomical enhancement garment 100, in accordance with one embodiment of the present invention. The anatomical enhancement garment 100 has an alternative base band 112 that is manufactured semi-rolled on the anatomical enhancement garment 100. The alternative base band 112 requires folding or rolling down in contrast to FIG. 1A which is manufactured in place. The internal silicon bead 140 is embedded directly onto the inside surface of the sheath portion 120 adjacent to the open aperture 130 of the anatomical enhancement garment 100. The elasticized security band 150 is shown attached at a single point on the lower portion of the open aperture 130. All other features of the anatomical enhancement garment 100 are the same as previously described in FIG. 1A and its descriptions.

FIG. 1C illustrates a side perspective view of a penis sheath 120 and separate thick band 110 at the base of an anatomical enhancement garment 100, in accordance with one embodiment of the present invention. The penis sheath 120 includes an internal embedded polymer bead 140. The internal embedded polymer bead 140 is made of silicon or any other suitable rubber like polymer compound. The silicon bead 140 or elasticized band 150 (FIG. 1B) is positioned behind the penis head PH after the penis is received through the front opening of the aperture 130 from inside the same at the opposite end. The internal embedded polymer bead 140 or elasticized security band 150 (FIG. 1B) disposed adjacent inside of the frontal aperture 130 provides the added security and non-slip characteristics needed to prevent unintentional retraction of the penis P back inside the sheath portion 120 when it is worn by the user. The thick band 110 can be used as a standalone piece or in combination with the penis sheath 120. In either case the thick band 110 is simply placed entirely over and around the genitals and then secured at the base where they attach next to the body. This embodiment does not include the pouch 160 component or its upper portion whereby it forms the thinner telescoping section 122 of the anatomical enhancement garment 100. Further additional embodiments might include only the penis sheath portion 120 of the garment and the silicon embedded polymer bead 140 without the base band 110, telescoping section 122 or pouch 160 components. The anatomical enhancement garment FIG. 1C in such case is essentially used as a simple penis extender that employs the displace and replace principals already described for modest augmentation and continued extension exercising of the penis tissue. In another embodiment the penis sheath 120, thick band 110 and furthermore the entire garment can be made of a latex rubber or similar type soft polymer compound to form a waterproof version of the said cloth fabric garment as described in the previous embodiments. These waterproof versions can be used for example when the user is wearing Speedo type swimwear during which cold water causes the testicles to retract and scrotum to shrink drastically. Under these more extreme environmental conditions the full latex versions not only provide for a highly adaptable and continuous fit in direct relation to the changing sizes of the said genitalia but also offer excellent insulating properties to minimize the extreme physiological effects of retraction and shrinking that are induced by such conditions on the male anatomy.

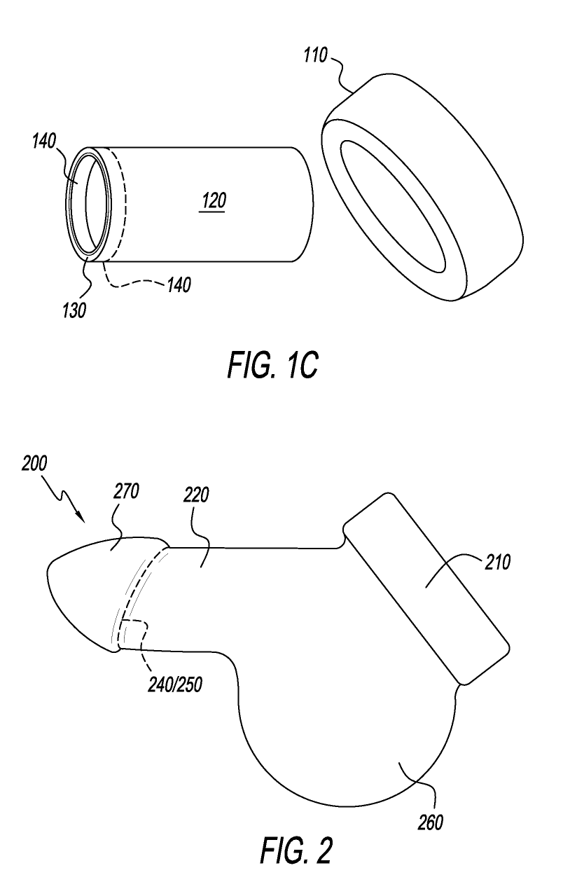

FIG. 2 illustrates a side perspective view of an anatomical enhancement garment 200, in accordance with one embodiment of the present invention. The anatomical enhancement garment 200 includes a base band 210, a penis sheath 220, a recessed internally embedded silicon bead 240 or elasticized security band 250, a pouch 260 and a glans cover 270.

The base band 210, the penis sheath 220 and the pouch 260 are the same as previously described in FIG. 1A and its description. The recessed internally embedded silicon bead 240 or elasticized band 250 provides additional securing functions and while maintaining comfort to the inserted penis P. The glans cover 270 envelopes the penis head PH as the penis P is inserted into the anatomical enhancement garment 200 through the penis sheath 220 and then through the recessed internal silicon bead 240 or elasticized band 250. The glans cover 270 can be made of cloth, latex or any other suitable material of which may also include a discrete opening for urination.

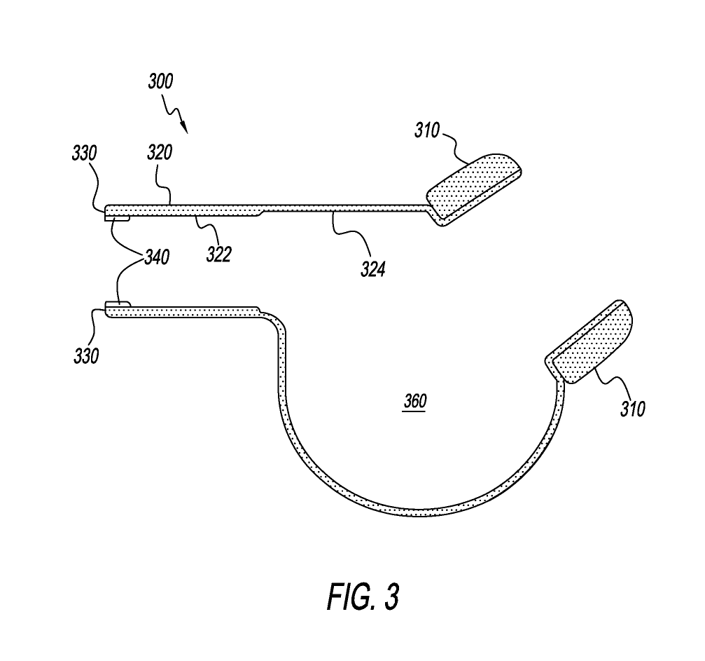

FIG. 3 illustrates a side perspective view of an anatomical enhancement garment 300, in accordance with one embodiment of the present invention. The anatomical enhancement garment 300 includes a base band 310, a penis sheath 320, a frontal aperture 330, an internal embedded silicon polymer type bead 340, and a pouch 360. The base band 310, the frontal aperture 330 and the pouch 360 are the same as previously described in FIG. 1A and FIG. 2 and their descriptions. The penis sheath 320 is illustrated showing the relatively thick portion 322 and a relatively thin portion 324. The relatively thick portion 322 starts at the frontal aperture 330 and narrows into the relatively thin portion 324, which ends at the base band 310. The relatively thin portion 324 can fold in an accordion like fashion as the penis P within the penis sheath 320 expands and contracts. The internal embedded polymer bead 340 is made of silicon or any similar acting rubber like compound to provide secure placement while maintaining the needed comfort for the inserted penis P.

In some cases, if needed a tong like instrument (not shown) can also be inserted into the anatomical enhancement garment 300 via the frontal aperture 330 then run down through the penis sheath 320 and out the band base 310. The tong is then firmly clamped over the penis head and the penis shaft stretched and made thin to help get the penis into position while donning the anatomical enhancement garment 300. Once the penis head is fully extended thru the frontal aperture 330 the clamped tong is then released and removed from the penis head after it is has been guided completely through the anatomical enhancement garment 300 which is then secured as usual in position at the base of the genitals next to the body.

The anatomical enhancement garment is generally speaking based on the traditional sock form, material and manufacture, although it may be made from panel type construction methods or a combination of the two techniques. In any case the fabric that usually covers the lower calf of the leg is rolled or folded down where it terminates at the heel of the sock thereby forming a base band of material where the garment is secured in place and forms a handle for gripping to don the garment. This folding action allows the material to be unraveled and thus fully extended to aid in the washing and rapid drying of the garment.

The anatomical enhancement garment can be manufactured to specific size configurations, which eliminates the need to roll the fabric down to fit and still act as a handle for gripping. Regardless, the scrotum and testicles sit in the heel of the garment which effectively acts as a pouch. The foot portion of the sock of the garment is manufactured or modified to form a smaller diameter tube and short length of thicker material which forms a penis sheath with a frontal aperture that acts as a passage way for the penis. The penis is additionally secured behind the glans with a silicon polymer bead embedded directly onto the surface of the inside portion of the open aperture of the garment to provide a no slip surface to secure the garment comfortably in place. Alternatively an elasticized band attached at the bottom portion of the open aperture of the penis sheath can be used to serve the same purpose.

If the elasticized band is used it may be implemented in any number of ways from a completely concealed design to a semi-concealed or entirely exposed design with an attachment at a single or multiple points at the outermost edge of the frontal aperture. This may also include a drawstring or clasp assembly, hook and loop fasteners, buttons, snaps or any other garment connecting device that contributes to a more convenient method of keeping the garment securely in place. The elasticized band may also be recessed inside the penis sheath to provide the extra length of material required to cover the entire penis glans.

The garment is designed with a proportionately thicker section of material strategically located along the penis shaft to create a firm penis sheath. This provides various degrees of extension capabilities to the penis while the thinner portion between the sheath and the thick band is able to retract and fold within itself in a telescopic or accordion like manner. In this way the garment is able to control and maintain the optimum stretched length of the penis while still allowing a certain degree of compensation for the natural size fluctuations of the penis.

A complete range of material thicknesses and densities will be used depending on the particular size gains desired to create a full product line of anatomical enhancement garments that will satisfy a wide variety of individual preferences. This will include various lengths of sheath and different thickness of material for additional size and support. The use of clips, snaps or zippers that are structurally integrated into the thick base band may also be used in some designs if needed to conveniently assist in getting in and out of the garment. Additionally the frontal aperture of the penis sheath can be tapered for greater form fitting which can be of a totally closed design in some applications. The elasticized band located at the frontal aperture can simply be attached at a strategic point along the outermost edge at the bottom and remain freely suspended for ease of placement behind the penis head after the garment is positioned. The relatively thicker base band may be permanently configured to size rather than rolled or folded down to fit.

The anatomical enhancement garment may be composed of a wide variety of natural and synthetic fiber blends to provide a full range of thermal, absorption, texture, elastic and odor control properties. Designs can also incorporate logos and trademarks using a wide spectrum of colors and a variety of trendy graphics including the use of catchy phrases. The ability to urinate is not compromised in any way while wearing the anatomical enhancement garment. A fully erect penis is also comfortably accommodated if a penis becomes erect. The anatomical enhancement garment is designed so it can be worn at night while sleeping without incident. It can easily be worn over both circumcised and uncircumcised penises.

The anatomical enhancement garment for men is an all-in-one approach to male genital support, augmentation and penis exercise for the male genitals as outlined in the following eight ways.

Firstly, there is the relatively thick elasticized band of material that is formed when the material is folded down to the specific size needed to fit the individual's genitals at the base and a second tubular cylinder or “sheath” of material that is fitted along the penis shaft. At both of these points the anatomical enhancement garment displaces and replaces the genital tissue for maximum size gains.

Secondly, the relatively thick band secures the anatomical enhancement garment in place to maintain a proper fit.

Thirdly, the relatively thick band conveniently acts as an effective handle for gripping the anatomical enhancement garment with the fingers to don the garment.

Fourthly, there is a design specific capacity for accurate variation of the garment's telescoping and accordion like action which compensates for the natural size fluctuations of a user's penis.

Fifthly, the open frontal aperture is secured with an embedded silicon bead bonded directly onto the fabric or uses instead an elasticized band ether of which is fitted directly behind the penis glans to prevent the penis from slipping unintentionally back inside the sheath.

Sixth, the manufacturing technique of adapting to the existing machinery used in standard sock production to take advantage of the inexpensive and speedy manufacturing systems already available for production of the garment which may or may not need to be additionally sewn and altered.

Seventh, the garment is designed in such a way that the user is able to put it on and take it off with remarkable ease and in less common anatomical configurations where it is not i.e., a very small penis in combination with very large fingers, the clamping tool effectively facilitates the same. The ability to urinate is not compromised in any way. The stretchable fabric accommodates the fully erect penis in both style and comfort. The garment is intended for usage around the clock, day in and day out over a period of years or even over the lifetime of the wearer. It can be worn at all times except when bathing or during sex.

Eighth, the garment can be manufactured from a soft latex type polymer compound to provide a waterproof thermo barrier which can be used, for example, while swimming. This material offers effective protection to minimize excessive shrinkage of the male genitalia due to the colder temperatures experienced while participating in such activities.

Any remotely similar products to the anatomical enhancement garment are limited to the lingerie or fetish wear lines which are intended exclusively for display purposes to generate arousal in the user's sexual partner. They are not for designed for use in any form of continuous support, augmentation and passive genital exercising applications.

Any penis exercising devices currently available in the marketplace are strictly mechanical and used intensively in active penis exercise programs of short duration. They are not intended for long term and continuous daily usage for passive genital size gains that include the scrotum. The anatomical enhancement garment complements those users already engaging in such active penis exercise programs to provide further continuous extension of genital tissue benefits 24 hours a day, 7 days a week.

The anatomical enhancement garment for men effectively reduces and can even eliminate the psychological symptoms experienced by those suffering from small penis syndrome. The anatomical enhancement garment enhances not only the size and mass of the male genital form but also the mental state of the wearer in two ways.

Firstly, the garment immediately produces the sensation and appearance of a much larger genital mass with a stretchable form fitting sock like knit fabric to envelope and contain the genitals to support and augment their size. The garment acts very much like a form enhancing brassiere for females looking to support and augment their natural size and shape. The male enhancement garment similarly provides size and shape enhancement for a man and also ensures that the male anatomy is neatly organized, tidy and secure to effectively give a man more confidence in essentially the same way as a bra does for a woman.

Secondly, the garment exercises tissue structures by passively training the penis and scrotum to remain in a fully extended condition. This is achieved by the positive stretching forces the garment applies to produce significant length gains in the flaccid state. These gains are initially temporary but with continuous full time and long term use these gains have proven to remain for relatively longer and longer periods. There are also indications that noticeable permanent flaccid gains are also possible with full time use of the garment.

The anatomical enhancement garment also works extremely well in the powerful realm of the “felt sense”. This is a term used to describe the experience of sensation in one's body directly before one has the chance to think about it. In this way the garment circumvents conscious thought and triggers specific neuro-emotional responses that produce powerful secondary “feel good” sensations. These positive life affirming sensations automatically resolve the negative psycho-emotional complexes that classically manifest around small penis syndrome complexes. This initiates the auto-immune response and a state of general well being is thus stimulated for healthy mental and physical life expression. For those with chronic deeply rooted psychological problems the garment has proven to help resolve these complexes at a fundamental level especially when used in conjunction with proven psycho-therapeutic healing processes including hypnosis.

The use of the anatomical enhancement garment for men provides optimized male anatomical support, augmentation and a passive exercising system to produce the immediate feeling of a more substantial genital mass. Actual size gains in the flaccid state are also temporarily possible and with long term usage for generating much longer lasting flaccid size gains which can become permanent if the garment is worn consistently thereafter. This leads to a significantly greater psychological confidence and a more physically relaxed body language in the male user.

These benefits automatically reduce the impact of the insecurity complexes that are related to feelings of inadequacy in men who perceive a deficiency with their flaccid proportions. Such complexes are naturally resolved without conscious effort simply as a result of wearing the garment because of its ability to generate the sensation of abundant fullness in the user with eventually longer lasting results from the extension training. This reduces the tendency of the penis to retract or “turtle” and furthermore leads to potentially permanent size gains.

Persons in the modeling and fashion industry may want to take advantage of the more accentuated and prominent look produced by the anatomical enhancement garment. Bodybuilders will also find the anatomical enhancement garment to be an excellent choice to augment their genital mass and presentation style particularly during competition or even casually while training in the gym. Those involved in any sport will appreciate the tidy support and augmentation properties that the anatomical enhancement garment provides. The garment can be worn under everyday clothing such as jeans, pants, shorts and swimwear and can be used by any man who wishes to not only support but also enhance and even permanently increase his genital proportions.

The anatomical enhancement garment offers 9 ways to achieve unparalleled male genital support and augmentation benefits by the following means:
1. The additional bulk of material the garment provides.
2. The use of bands to control blood flow to keep the organ semi-engorged.
3. The use of a telescopic and accordion like action that results in a continuous penis fit.
4. The garment maintains a higher temperature around the genitals thus diminishing the tendency of a penis to shrink excessively.
5. The garment pulls the genital mass into an anterior and superior position for more prominent display.
6. The garment provides excellent support to prevent the genitals from slipping between the legs or getting stuck-up in awkward and uncomfortable positions which can contribute to a diminished appearance.
7. The garment prevents the tendency of the penis to “turtle”, which is a condition where the penis withdraws itself inwards toward the body in an extremely diminished flaccid state.
8. The garment passively exercises the genital tissues in such a way that temporary gains in mass and volume are realized immediately. These gains become increasingly longer lasting with continuous usage over an extended period of time.
9. The garment uses displace and replace principles which are employed at two points that adds volume to the genital mass and extends the penis length and add penis girth.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A male anatomical enhancement garment to enhance male genitalia with a base, a penis, a scrotum and testicles and a penis head, comprising:
a base band to receive said genitalia that wraps around said base of said genitalia to secure said garment in place;
a penis sheath with an end adjacent to said base band to receive said penis that expands and contracts as said penis expands and contracts and ;
a frontal aperture disposed on said end of penis sheath that said inserted penis is inserted through;
a silicon bead directly embedded inside of said frontal aperture that is positioned behind said penis head after said penis is received through said front aperture;
an elasticized band disposed inside of said frontal aperture that is positioned behind said penis head after said penis is received through said front aperture; and
a pouch that contains said scrotum and testicles and is adjacent to said base band.

2. The garment according to claim 1, wherein said base band is formed by folding or rolling down material to fit said genitalia.

3. The garment according to claim 1, wherein said base band is manufactured to size and permanently affixed to said garment.

4. The garment according to claim 1, wherein said penis sheath is made of material that expands and contracts as said penis expands and contracts.

5. The garment according to claim 1, wherein said penis sheath includes an internal embedded bead of silicon type polymer.

6. The garment according to claim 1, wherein said penis sheath includes an internal attached elasticized band.

7. The garment according to claim 1, wherein said garment is made of cloth fabric.

8. The garment according to claim 1, wherein said garment is made of latex type polymer.

9. A male anatomical enhancement garment to enhance male genitalia with a base, a penis, a scrotum and testicles and a penis head, comprising:
a base band to receive said genitalia that wraps around said base of said genitalia to secure said garment in place;
a penis sheath with an end adjacent to said base band to receive said penis that expands and contracts as said penis expands and contracts;
a frontal aperture disposed on said end of penis sheath that said inserted penis is inserted through;
an internal embedded polymer bead disposed in front of said frontal aperture that is positioned behind said penis head after said penis is received through said front aperture;
an elasticized band disposed in front of said frontal aperture that is positioned behind said penis head after said penis is received through said front aperture; and
a pouch that contains said scrotum and testicles that is adjacent to said base band.

10. The garment according to claim 9, wherein said base band is folded or rolled to fit said genitalia or is fitted with snaps, buttons or zippers to fit said genitalia.

11. The garment according to claim 9, wherein said base band is permanently affixed to said garment.

12. The garment according to claim 9, wherein said penis sheath is made of material that expands and contracts as said penis expands and contracts.

13. The garment according to claim 9, wherein said penis sheath includes an internal embedded polymer bead adjacent inside of the frontal aperture.

14. The garment according to claim 9, wherein said penis sheath includes an elasticized band adjacent to the frontal aperture.

15. The garment according to claim 9, wherein said garment is made of material selected from the group consisting of cloth, a latex material or a similar soft polymer type compound.

16. The garment according to claim 9, wherein said internal embedded bead is selected from the group consisting of silicon, PU, thermoplastic, rubber, or a rubber like polymer compound.

17. A male anatomical enhancement garment to enhance male genitalia with a base, a penis, a scrotum and testicles and a penis head, comprising:
a base band to receive said genitalia that wraps around said base of said genitalia to secure said garment in place;
a penis sheath with an end adjacent to said base band to receive said penis that expands and contracts as said penis expands and contracts;
a recessed internal band disposed on said end of penis sheath that provides additional securing and comfort to said inserted penis;
a pouch that contains said scrotum and testicles that is adjacent to said base band; and
a glans cover that covers said penis head.

18. The garment according to claim 17, wherein said base band is permanently affixed to said garment.

19. The garment according to claim 17, wherein said penis sheath includes an internal embedded polymer bead.

20. The garment according to claim 17, wherein said garment and said gland cover is selected from the group consisting of cloth material, latex material or a similar soft polymer type compound.
